(19) United States
(12) Patent Application Publication
(10) Pub. No.: US 2025/0268967 A1
(43) Pub. Date: Aug. 28, 2025

(54) NUTRACEUTICAL COMPOUNDS USEFUL IN THE TREATMENT OF CORONAVIRUS DISEASE

(71) Applicants: SKYMOUNT MEDICAL US INC., New Orleans, LA (US); THE BOARD OF SUPERVISORS OF LOUISIANA STATE UNIVERSITY AND AGRICULTURAL AND MECHANICAL COLLEGE, Baton Rouge, LA (US)

(72) Inventors: Adam BESS, Baton Rouge, LA (US); Frej Knut, Gosta BERGLIND, Baton Rouge, LA (US); Supratik MUKHOPADHYAY, Baton Rouge, LA (US); Kishor M. WASAN, Saskatoon, Saskatchewan (CA); Chris GALLIANO, New Orleans, LA (US); Michal BRYLINSKI, Baton Rouge, LA (US); Stephania CORMIER, Baton Rouge, LA (US); Allan ADER, New Orleans, LA (US); Nicholas GRIGGS, New Orleans, LA (US); Janet GOULD, New Orleans, LA (US); Tiffany CHO, New Orleans, LA (US); Julia ABRAMOV, New Orleans, LA (US); Peter HNIK, New Orleans, LA (US)

(73) Assignees: SKYMOUNT MEDICAL US INC., New Orleans, LA (US); THE BOARD OF SUPERVISORS OF LOUISIANA STATE UNIVERSITY AND AGRICULTURAL AND MECHANICAL COLLEGE, Baton Rouge, US (LA)

(21) Appl. No.: 18/268,146

(22) PCT Filed: Dec. 17, 2021
(86) PCT No.: PCT/US2021/073019

§ 371 Date: Jun. 16, 2023

Related U.S. Application Data
(60) Provisional application No. 63/168,791, filed on Mar. 31, 2021.

(30) Foreign Application Priority Data
Dec. 18, 2020 (CA) 3102947

(51) Int. Cl. A61K 36/258; A61K 31/045; A61K 31/07; A61K 31/355; A61K 31/365; A61K 31/375; A61K 31/593; A61K 31/7076; A61K 31/7084; A61K 33/30; A61P 31/14

(52) U.S. Cl. CPC A61K 36/258; A61K 31/045; A61K 31/07; A61K 31/355; A61K 31/365; A61K 31/375; A61K 31/593; A61K 31/7076; A61K 31/7084; A61K 33/30; A61P 31/14

(57) ABSTRACT

The present disclosure relates to use of and a method of administering a formulation that comprises a combination of at least two compounds selected from the group consisting of: levomenol, a ginseng extract, ascorbic acid; adenine derivatives (Adenosine; NAD+/NADH); ginkgolides such as ginkgolide A; and vitamin E; for treating one or more symptoms of a SARS-CoV-2 infection and for ameliorating symptoms that may persist following a SARS-CoV-2 infection.

### TECHNICAL FIELD

The present invention is directed to a nutraceutical formulation that may be useful in treatment of infections. In particular, the nutraceutical formation may be useful in treating or mitigating symptoms of a coronavirus infection.

### BACKGROUND

The appearance of a novel coronavirus, referred to as SARS-CoV-2, on the world stage has affected substantially every population in the world. This virus has afflicted millions of individuals and caused a disease, referred to as COVID-19. COVID-19 can develop into a significant health risk and result in death, which has placed a high strain on healthcare resources and society in general.

SARS-CoV-2 is a single-strand, positive-sense ribonucleic acid (RNA) virus with a similar receptor-binding domain structure to that of SARS-CoV and MERS-CoV. SARS-CoV-2 is transmitted between individuals via airborne droplets accessing nasal mucosa. Within the nasal mucosa SARS-CoV-2 can rapidly reproduce and be shed in nasal secretions (sputum). Sputum can be transmitted to other individuals via airborne droplets, thus repeating the transmission cycle. The SARS-CoV-2 virus can spread between individuals before the onset of symptoms, during the symptomatic period and even after recovery.

The clinical spectrum of the infection is wide, ranging from mild signs of an upper respiratory tract infection to severe pneumonia, multi-organ failure and death. At the onset, SARS-CoV-2 primarily attacks the respiratory system, as it represents the main point of entry into the host, but SARS-CoV-2 also can affect multiple organs of an infected individual. The severity of COVID-19 is typically associated with comorbidities such as, but not limited to: hypertension, diabetes, obesity, and/or advanced age that can exacerbate the consequences of COVID-19.

A number of vaccines for SARS-CoV-2 have become available and form an important part of health official's recommendations to ease the COVID-19 public health crisis and to manage the stress COVID-19 has placed on health care workers and hospitals. However, the SARS-CoV-2 virus has already demonstrated the ability to mutate into various different variants, some of which are more transmissible than other variants and some of which are proving successful at avoiding the protections afforded by current vaccines.

As such, there exists a need for further therapies that are capable of mitigating the personal health and public health impact of SARS-CoV-2 and its known and not yet known variants.

### SUMMARY

The embodiments of the present disclosure relate to formulations that comprises two or more nutraceutical compounds for administering to a subject that may be or has been infected with a SARS-CoV-2 virus for: reducing the. In some embodiments of the present disclosure, the two or more nutraceutical compounds comprise levomenol and a ginseng extract. In some embodiments of the present disclosure, the two or more nutraceutical compounds comprise levomenol, a ginseng extract and zinc.

In some embodiments of the present disclosure, the two or more nutraceutical compounds comprise levomenol, a ginseng extract, zinc and vitamin D.

Some embodiments of the present disclosure relate to a formulation for delivery of a therapeutically effective amount of levomenol and a ginseng extract, wherein the formulation comprises at least 20% (by weight) of levomenol and at least 20% of a ginseng extract.

Some embodiments of the present disclosure relate to use of at least two compounds selected from the group consisting of: levomenol; zinc; calcium; beta-carotene; vitamin A; vitamin E; vitamin D; and ginseng extract for mitigating and/or substantially preventing a SARS-CoV-2 infection in a subject.

Some embodiments of the present disclosure relate to use of one or more or all of levomenol; zinc, vitamin D; and a ginseng extract for treating SARS-CoV-2 infection and/or post-COVID-19 symptoms in a subject.

Some embodiments of the present disclosure relate to use of one or more or all of levomenol; zinc, vitamin D; and a ginseng extract for mitigating and/or substantially preventing a SARS-CoV-2 infection in a subject.

Some embodiments of the present disclosure relate to use of one or more or all of: ascorbic acid; adenine derivatives (Adenosine; NAD+/NADH); a ginseng extract; and vitamin E; for mitigating and/or substantially preventing a SARS-CoV-2 infection in a subject.

Some embodiments of the present disclosure relate to use one or more or all of: ascorbic acid; adenine derivatives (Adenosine; NAD+/NADH); a ginseng extract; and vitamin E; for treating SARS-CoV-2 infection and/or post-COVID-19 symptoms in a subject.

Some embodiments of the present disclosure relate to use of one or more or all of: levomenol; zinc; calcium; beta-carotene; vitamin A; vitamin E; vitamin D; and a ginseng extract; ascorbic acid; adenine derivatives (Adenosine; NAD+/NADH); ginkgolides such as ginkgolide A; and vitamin E; for the treatment of post-COVID-19 symptoms in a mammal.

Some embodiments of the present disclosure relate to an oral dosage form comprising: levomenol; zinc, vitamin D; and a ginseng extract for use in preventing SARS-CoV-2 infection, one or more symptoms of COVID-19 or one or more symptoms following a SARS-CoV-2 infection.

Some embodiments of the present disclosure relate to a method of preventing infection by SARS-CoV-2 virus, the method comprising the steps of: administering a daily therapeutically effective amount of levomenol and a ginseng extract to a subject.

Some embodiments of the present disclosure relate to a method of preventing infection by SARS-CoV-2 virus, the method comprising the steps of: administering a daily therapeutically effective amount of levomenol, a ginseng extract and zinc to a subject.

Some embodiments of the present disclosure relate to a method of preventing infection by SARS-CoV-2 virus, the method comprising the steps of: administering a daily therapeutically effective amount of levomenol, a ginseng extract, zinc and vitamin D to a subject.

### BRIEF DESCRIPTION OF THE DRAWINGS

These and other features of the present disclosure will become more apparent in the following detailed description in which reference is made to the appended drawings.

FIG. 1 shows percent inhibition in vitro data from control compound.

FIG. 2 shows percent inhibition in vitro data from a nutraceutical compound of the present disclosure, relative to the control compound.

FIG. 3 shows percent inhibition in vitro data from a nutraceutical compound of the present disclosure, relative to the control compound.

FIG. 4 shows viral titer and cell viability in vitro data over various doses of the nutraceutical compound of FIG. 3.

FIG. 5 is a histogram that shows in vitro data of TCID50/ml observed in VERO6 cells treated with one of the nutraceutical compounds of the present disclosure.

FIG. 6 shows percent inhibition in vitro data from a nutraceutical compound of the present disclosure, relative to the control compound.

### DETAILED DESCRIPTION

Unless defined otherwise, all technical and scientific terms used herein have the meanings that would be commonly understood by one of skill in the art in the context of the present description. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

As used herein, the singular forms “a”, “an”, and “the” include plural references unless the context clearly dictates otherwise. For example, reference to “an agent” includes one or more agents and reference to “a subject” or “the subject” includes one or more subjects.

As used herein, the terms “about” or “approximately” refer to within about 25%, preferably within about 20%, preferably within about 15%, preferably within about 10%, preferably within about 5% of a given value or range. It is understood that such a variation is always included in any given value provided herein, whether or not it is specifically referred to.

As used herein, the term “activity” is used interchangeably with the term “functionality” and both terms refer to the physiologic action of a biomolecule.

As used herein, the term “agent” refers to one or more the nutraceutical compounds of the present disclosure that, when administered to a subject, cause one or more chemical reactions and/or one or more physical reactions and/or or one or more physiological reactions and/or one or more pharmacological reactions and/or one or more immunological reactions in the subject. Wherein each individual nutraceutical compound is considered an agent.

As used herein, the term “ameliorate” refers to improve and/or to make better and/or to make more satisfactory.

As used herein, the term “cell” refers to a single cell as well as a plurality of cells or a population of the same cell type or different cell types. Administering an agent to a cell includes in vivo, in vitro and ex vivo administrations and/or combinations thereof.

As used herein, the term “complex” refers to an association, either direct or indirect, between one or more particles of an agent and one or more target cells or target virions. This association results in a change in the metabolism or functionality of the target cells or target virions. As used herein, the phrase “change in metabolism” refers to an increase or a decrease in the one or more of the targets' production of one or more proteins, and/or any post-translational modifications of one or more proteins. As used herein, the phrase “change in functionality” refers to a difference in physiological function of one or more aspects of the target within an agent/target complex as compared to a target that is not part of such a complex.

As used herein, the term “compound” refers to a chemical compound with a known formula and all enantiomers, diastereomers, racemates, tautomers, or metabolite thereof, and all pharmaceutically acceptable salts, solvates or hydrates of the compound, enantiomers, diastereomers, racemates, tautomers, or metabolites thereof.

As used herein, the terms “dysregulation” and “dysregulated” refer to situations or conditions wherein homeostatic control systems have been disturbed and/or compromised so that one or more metabolic, physiologic and/or biochemical systems within a subject operate partially or entirely without said homeostatic control systems.

As used herein, the term “excipient” refers to any substance, not itself an agent, which may be used in a composition for delivery of one or more agents, and the like to a subject or alternatively combined with one or more carriers and the like (e.g., to create a pharmaceutical composition) to improve its handling or storage properties or to permit or facilitate formation of a dose unit of the composition (e.g., formation of a topical hydrogel which may then be optionally incorporated into a transdermal patch). Excipients include, by way of illustration and not limitation, binders, disintegrants, taste enhancers, solvents, thickening or gelling agents (and any neutralizing agents, if necessary), penetration enhancers, solubilizing agents, wetting agents, antioxidants, lubricants, emollients, substances added to mask or counteract a disagreeable odor, fragrances or taste, substances added to improve appearance or texture of the composition and substances used to form the pharmaceutical compositions. Any such excipients can be used in any dosage forms according to the present disclosure. The foregoing classes of excipients are not meant to be exhaustive but merely illustrative.

As used herein, the terms “inhibit”, “inhibiting”, and “inhibition” refer to a decrease in activity, response, or other biological parameter of a biologic process, disease, disorder or symptom thereof. This can include but is not limited to the complete ablation of the activity, response, condition, or disease. This may also include, for example, a 10% reduction in the activity, response, condition, or disease as compared to the native or control level. Thus, the reduction can be a 10%, 20%, 30%, 40%, 50%, 60%, 70%, 80%, 90%, 100%, or any amount of reduction in between the specifically recited percentages, as compared to native or control levels.

As used herein, the term “medicament” refers to a medicine and/or pharmaceutical composition that comprises an agent and that can promote recovery from a disease, disorder or symptom thereof and/or that can prevent a disease, disorder or symptom thereof and/or that can inhibit the progression of a disease, disorder, or symptom thereof.

As used herein, the term “pharmaceutical composition” means any composition comprising, but not necessarily limited to, one or more agents to be administered a subject in need of therapy or treatment of a disease, disorder or symptom thereof. Pharmaceutical compositions may include additives such as pharmaceutically acceptable carriers, pharmaceutically accepted salts, excipients and the like. Pharmaceutical compositions may also additionally include one or more further active ingredients such as antimicrobial agents, anti-inflammatory agents, anaesthetics, analgesics, and the like.

As used herein, the term “acceptable carrier” refers to an essentially chemically inert and nontoxic component within a pharmaceutical composition or medicament that does not inhibit the effectiveness and/or safety of the one or more agents. Some examples of pharmaceutically acceptable carriers and their formulations are described in Remington (1995, The Science and Practice of Pharmacy (19th ed.) ed. A. R. Gennaro, Mack Publishing Company, Easton, PA), the disclosure of which is incorporated herein by reference. Typically, an appropriate amount of a pharmaceutically acceptable carrier is used in the formulation to render said formulation isotonic.

Examples of suitable pharmaceutically acceptable carriers include, but are not limited to: saline solutions, glycerol solutions, ethanol, N-(1(2, 3-dioleyloxy)propyl)-N,N,N-trimethylammonium chloride (DOTMA), dioleolphosphotidylethanolamine (DOPE), and liposomes. Such pharmaceutical compositions contain a therapeutically effective amount of the agent, together with a suitable amount of one or more pharmaceutically acceptable carriers and/or excipients so as to provide a form suitable for proper administration to the subject. The formulation suits the route of administration. For example, oral administration may require enteric coatings to protect the agent from degrading within portions of the subject's gastrointestinal tract. In another example, injectable routes of administration may be administered in a liposomal formulation to facilitate transport throughout a subject's vascular system and to facilitate delivery across cell membranes of targeted intracellular sites.

As used herein, the phrases “prevent”, “prevention of” and “preventing” refer to avoiding the onset or progression of a disease, disorder, or a symptom thereof.

As used herein, the term “subject” refers to any therapeutic target that receives the agent. The subject can be a vertebrate, for example, a mammal including a human. The term “subject” does not denote a particular age or sex. The term “subject” also refers to one or more cells of an organism, an in vitro culture of one or more tissue types, an in vitro culture of one or more cell types, ex vivo preparations, and/or a sample of biological materials such as tissue and/or biological fluids.

As used herein, the term “target cell” refers to one or more cell types within a subject that can interact with a coronavirus by the virus fusing with the outer membrane of the one or more cell types, entering into the cell and/or replicating therein. Without being bound to any particular theory, target cells of a subject can include any cells within a subject that express the receptors and/or cofactors required for viral interaction. Examples of these types of cells include, but are not limited to: epithelial cells of the upper airways and conducting airways (ciliated and non-ciliated); alveolar epithelial cells (both type 1 and 2); epithelial cells and neurons of the olfactory system; neurons of the central or peripheral nervous system; epithelial cells, enteroctytes and gland cells of the gastrointestinal tract; cells of the blood, including immune effector cells; cardiovascular cells; and, renal cells.

As used herein, the term “target virion” refers to one or more viral particles of coronavirus that have the capacity to cause a viral infection within a target cell. In some embodiments of the present disclosure, the viral particles are of one or more variants of SARS-CoV-2.

As used herein, the term “therapeutically effective amount” refers to the amount of the agent used that is of sufficient quantity to ameliorate, prevent, treat and/or inhibit one or more of a disease, disorder or a symptom thereof. The “therapeutically effective amount” will vary depending on the agent used, the route of administration of the agent and the severity of the disease, disorder or symptom thereof. The subject's age, weight and genetic make-up may also influence the amount of the agent that will be a therapeutically effective amount.

As used herein, the terms “treat”, “treatment” and “treating” refer to obtaining a desired pharmacologic and/or physiologic effect. The effect may be prophylactic in terms of completely or partially preventing an occurrence of a disease, disorder or symptom thereof and/or the effect may be therapeutic in providing a partial or complete amelioration or inhibition of a disease, disorder, or symptom thereof. Additionally, the term “treatment” refers to any treatment of a disease, disorder, or symptom thereof in a subject and includes: (a) preventing the disease from occurring in a subject which may be predisposed to the disease but has not yet been diagnosed as having it; (b) inhibiting the disease, i.e., arresting its development; and, (c) ameliorating the disease.

As used herein, the terms “unit dosage form” and “unit dose” refer to a physically discrete unit that is suitable as a unitary dose for patients. Each unit contains a predetermined quantity of the agent and optionally, one or more suitable pharmaceutically acceptable carriers, one or more excipients, one or more additional active ingredients, or combinations thereof. The amount of agent within each unit is a therapeutically effective amount.

In embodiments of the present disclosure, the pharmaceutical compositions disclosed herein comprise one or more agents as described above in a total amount by weight of the composition of about 0.1% to about 95%. For example, the amount of the agent by weight of the pharmaceutical composition may be about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, about 90% or about 95% or more.

Where a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also, encompassed within the disclosure, subject to any specifically excluded limit in the stated range. Where the stated range includes one or both of the limits, ranges excluding either or both of those included limits are also included in the disclosure.

The appearance of COVID-19 on the world stage has affected most if not all populations in the world. Causing millions of infected individuals, a number which is continuously increasing and is showing no signs of slowing down, in spite of multiple vaccines being available.

In light of this, it is important to investigate treatments and possibly treatment compositions that are based on widely available and approved compounds to help prevent SARS-CoV-2 infection and/or treat the symptoms of individuals experiencing or recovering from a SARS-CoV-2 infection. The embodiments of the present disclosure relate to treatment compositions, such as nutraceutical formulae, that are designed to help with various vitamin/mineral imbalances that may be caused by a SARS-CoV-2 infection.

The embodiments of the present disclosure relate to nutraceutical formulations that were designed and then investigated to determine their potential to treat SARS-CoV-2 infection and its symptoms. Seven nutraceutical constituent compounds were retained for their potential use for treatment of COVID-19 and/or as a potential preventative treatment for protection from SARS-CoV-2 infection by immune system stimulation. A review of artificial intelligence (AI) scores, mechanisms of action, and known anti-viral, anti-inflammatory or immunomodulatory data for each compound was conducted.

According to some embodiments of the present invention, various nutraceutical ingredients may be used in combination with an active pharmaceutical ingredients (APIs), as part of a combination treatment for the treatment of COVID-19 infection or the treatment of symptoms related to a COVID-19 infection.

According to some embodiments of the present invention, levomenol and vitamin C appear to have a high potential in the treatment of SARS-CoV-2, based on: 1) a high AI score supported by pharmacology (Levomenol); and 2) extensive evidence in the prevention or treatment of viral infections that support its potential utility for treatment of SARS-CoV-2 (Vitamin C).

To the best of the inventor's knowledge, levomenol is not being considered currently as a treatment for SARS-CoV-2. If levenomol shows antiviral activity in vitro and if it can be formulated into an acceptable formulation separately or with the other nutraceuticals, the combined product could present a unique nutraceutical therapy for use in treating viral infections, such as SARS-CoV-2 infection.

According to other embodiments of the present invention, vitamin A; Folic acid and vitamin E may be used in combination therapy for either prevention (i.e. the immuno-stimulatory effects of vitamin E) or protection from viral infection (i.e. antiviral effects of folic acid) or both (vitamin A).

The embodiments of the present disclosure relate to the use of the nutraceutical compounds, as described further herein below, to: reduce or substantially prevent infection of a subject by a SARS-CoV-2 virus; treat, ameliorate or substantially reduce one or more symptoms of a SARS-CoV-2 virus; and, treat, ameliorate or substantially improve an imbalance of one or more vitamins and minerals in a subject who is or has been infected with a SARS-CoV-2 virus.

### Levomenol

Levomenol (also known as α-(−)-bisabolol) is used in decorative cosmetics, fine fragrances, shampoos, and other toiletries, as well as in non-cosmetic products such as household cleaners and detergents and also in pharmaceutical formulations. Levomenol belongs to the family of sesuiterpenoid compounds and it may be isolated from essential oil of Matricaria chamomilla (German chamomile). The main application of levomenol in pharmaceutical preparation is related to its anti-inflammatory, antispasmodic, anti-allergic, drug permeating, and vermifuge properties.

Chamomile essential oil has been used for centuries as an anti-inflammatory agent for alleviating symptoms associated with eczema, dermatitis, and other pronounced irritation. Volatile constituents present in the chamomile oil notably levomenol, exert anti-inflammatory activity partly due to inhibition of leukotriene synthesis. Levomenol is likely to be a 5-lipoxygenase (5-LOX) inhibitor with in vitro IC₅₀ values for this compound on 5-LOX ranging from 10 to 30 μg/ml. It is also worth noting that it has been established that the 5-LOX pathway is involved in the virus pathophysiology). In fact, both in infected animal models (mice) and in humans, exposure to influenza virus has shown to upregulate 5-LOX in the lungs. Leukotriene B4 (LTB4), which is a metabolite of the 5-LOX pathway, has shown to inhibit influenza viral replication. Additionally, LTB4-treated neutrophils have demonstrated enhanced virucidal activity against influenza, human coronavirus, and respiratory syncytial virus (RSV). While the implications of the 5-LOX pathway in SARS-CoV-2 infection are not completely understood, based on the information summarized above, further investigation in relevant in vitro and in vivo preclinical assays are warranted to assess the efficacy of levomenol in treatment of patients with SARS-CoV-2 infections.

### Zinc

Zinc is an essential trace element, ubiquitously found in all living organisms. It is one of the most common elements in the Earth's crust and is found in soil, water, as well as all animal and plant-based foods. The primary dietary sources of zinc include seafood, especially oysters, which are the richest source of zinc (up to 1000 ppm), meats, and nuts. Zinc targets glycogen synthase kinase-3 beta (15 μM).

Zinc deficiency is associated with increased susceptibility to infectious diseases caused by bacterial, viral, and fungal pathogens, and may be caused by diseases such as liver cirrhosis or inflammatory bowel diseases, aging, and lifestyle-associated factors (e.g., vegan/vegetarian diets can be low in zinc).

The potential use of zinc supplementation in the prophylaxis and treatment of SARS-CoV-2 infection is currently being investigated in several clinical trials in both in the US and EU. In these studies, zinc, administered either in combination with other therapeutics or supplementations, is currently being evaluated in patients with SARS-CoV-2 infections at oral doses of 15 to 220 mg/day (administered as zinc, zinc sulfate, or zinc gluconate). In this study, wherein an oral dose of 220 mg/day was evaluated, doses were administered only for 5 days. While the use of zinc in the treatment of patients with SARS-CoV-2 infections is being extensively evaluated, there are certain caveats or restrictions associated with the use of zinc supplements. Firstly, doses greater than 150 mg/day are not recommended in adults. Additionally, in a randomized, double-bind, placebo-controlled clinical trial conducted in subjects with common cold, children less than 12 years of age did not appear to benefit following oral administration of zinc (given as zinc gluconate). Finally, the use of nasal zinc formulation is not recommended. In fact, in 2009, the US Food and Drug Administration (FDA) warned against such products because people who used them lost their sense of smell.

Without being bound by any particular theory, it was hypothesized that zinc supplementation may be of potential benefit for prophylaxis and treatment of SARS-CoV-2 infections, which may be achieved via several direct and indirect antiviral properties. For instance, in vitro studies have shown that zinc induces the production of IFN-α and IFN-γ and can potentiate the antiviral action of the former. Additionally, zinc supplementation in healthy adults has also been associated with a decreased production of TNF-α and IL-1. Zinc also enhances cellular resistance to apoptosis (programmed cell death) through inhibition of caspases-3, -6, and -9, and an increase of the Bcl-2/Bax ratio, and such anti-apoptotic effects at both the peripheral and thymic level could result in an increase in the number of T helper cells. Zinc-induced alteration of the capillary epithelium may inhibit transcapillary movement of plasma proteins and reduce local edema, inflammation, exudation, and mucus secretion. Finally, zinc may also protect or stabilize the cell membrane which may contribute to an inhibition of the virus entry into the cell. Antiviral effects of zinc may also be achieved through metallothioneins (MTs), a family of low molecular weight, cysteine-rich zinc-binding proteins with functions including storage and transfer of zinc. Previous studies have demonstrated that overexpression of multiple members of the MT1 family inhibits replication of flaviviruses (e.g., yellow fever virus and HCV), as well as the alphavirus (Venezuelan equine encephalitis virus).

In addition to the indirect antiviral mechanism, the direct antiviral properties of zinc against a several viral species involve several mechanisms including physical processes such as virus attachment, infection, and uncoating, as well as through enzymatic processes that involve inhibition of viral protease and polymerase enzymes. Zinc is considered crucial for the proper folding and activity of various cellular enzymes and transcription factors, and may also be an important co-factor for several virus proteins. In viruses such as picorna, encephalomyocarditis, and polio viruses, zinc may also interfere with the proteolytic processing of viral polyprotein by its misfolding, result in direct actions on the viral protease, and cause alterations of the tertiary structure. In respiratory syncytial virus, HSV, Semliki Forest virus and sindbis viruses, zinc has shown to efficiently inhibit membrane fusion, which is achieved via binding to a specific histidine residue revealed on the viral E1 protein at low endosomal pH. Finally, in in vitro studies, zinc has demonstrated a potential for direct inactivation of certain viruses such as the free Varicella-Zoster virus.

Following a review of the pharmacological profile for zinc, it is likely that zinc supplementation may be beneficial for the prophylaxis and treatment of COVID-19. There are several lines of evidence that support this claim. Firstly, zinc possesses several direct and indirect antiviral effects which can be achieved via generation of both innate and acquired immune responses, facilitation of the normal functioning of innate immune system, stabilization of cell membrane thereby inhibiting the entry of the virus, and inhibition of viral replication through interference with the viral genome transcription, protein translation, polyprotein processing, viral attachment, and uncoating. Secondly, the antiviral effects of zine has been demonstrated in several viral species, including several nidoviruses.

### Vitamin A and Vitamin A Palmitate

Vitamin A is a fat-soluble nutrient that can be found preformed in animals (e.g., fish liver oils, eggs, liver of most vertebrates, dairy products) and plant sources (e.g., carotenoids). Vitamin A can also be used as a dietary supplement in drugs and other consumables as it is generally recognized as a safe nutrient. Physiologically, it is essential for normal cellular proliferation and differentiation of epithelial tissue, embryonic development, bone growth, reproduction maintenance of the immune system, and good vision. Large oral doses of vitamin A are used to treat deficiency states and certain skin diseases at varying doses above the recommended dietary allowance (RDA).

Different forms of vitamin A are required by the body in multiple physiological processes. For example, retinal is required by the eye for proper vision and the retinoic acid form is needed for proper cell differentiation of the cornea and other ocular structures as well as proper maintenance of immune function.

Vitamin A is required for the integrity of epithelial cells, as it regulates the expression of numerous genes within the body that encode for structural proteins and enzymes. Retinoic acid receptors (RARs) and retinoid-X receptors (RXRs) are the transcription factors that modulate gene transcription; when there is a deficiency in vitamin A, natural cell differentiation and growth are interrupted.

Retinoids are a family of molecules that possess qualitative activity relative to vitamin A that can be used as potential immunomodulators against SARS-CoV-2, and can regulate the expression of genes involved in innate and adaptive immune responses. The combination of retinoid compounds and type I interferons (IFNs), such as IFN-α and IFN-0, has been suggested to synergistically potentiate immune-mediated antiviral effects against viruses. Retinol can also be converted into other active derivatives that aid host defense against acute infection.

### Vitamin C

Vitamin C is a water-soluble vitamin and is essential in humans whereas most animals can synthesize it. The term vitamin C refers to both compounds of ascorbic acid and dehydroascoribic acid (DHA). There are two enantiomeric forms, of which the L form is the naturally occurring active form; and the D-form, isoascorbic or erythorbic acid, provides antioxidant but little or no anti-scorbutic activity.

Vitamin C deficiency causes scurvy, which is characterized by fatigue or lassitude, widespread connective tissue weakness, and capillary fragility. The primary dietary sources in the typical diet of Vitamin C come from fruits and vegetables, with citrus fruits, tomatoes and tomato juice, and potatoes being the major contributors. Other sources include brussel sprouts, cauliflower, broccoli, strawberries, cabbage, and spinach. Vitamin C is also added to some processed foods as an antioxidant.

Vitamin C has been used to treat scurvy, macular degeneration, respiratory infection and sepsis. Although vitamin C has not been shown in well-controlled trials to have therapeutic value, it has been prescribed for hematuria, retinal hemorrhages, hemorrhagic states, dental caries, pyorrhea, gum infections, anemia, acne, infertility, atherosclerosis, mental depression, peptic ulcer, tuberculosis, dysentery, collagen disorders, cancer, osteogenesis imperfecta, fractures, leg ulcers, pressure sores, physical endurance, hay fever, heat prostration, vascular thrombosis prevention, levodopa toxicity, succinylcholine toxicity, arsenic toxicity, and as a mucolytic agent.

The potential use of Vitamin C supplementation in combination with other specific vitamin supplement or APIs is actively being tested in 37 clinical trials. Doses being administered include oral dosage of 8 g daily, given in 2 or 3 divided doses on an outpatient basis. For more severe patients, 1.5 g is administered every 6 hours until shock resolution or up to 10 days; or after an initial 200-mg/kg IV dose, 1 g orally 3 times daily for 7 days. The proposed dose to be administered in combination with quercetin is 500 mg/day Vitamin C for prophylaxis or mild cases, whereas, 3 grams 6 times per day for 7 days in severe cases.

The pharmacological mechanism of action of Vitamin C is as an electron donor and a reducing agent. It is hypothesized that Vitamin C supplementation in combination with other supplements or medicines may be of potential benefit for prophylaxis and treatment of COVID-19, which may be achieved via antioxidant and immunomodulatory properties.

### Vitamin D3 and Vitamin D2

Vitamin D (calciferol) refers to a series of compounds including vitamin D1 (a 1:1 mixture of vitamin D2 and lumisterol), vitamin D2 (ergocalciferol), vitamin D3 (cholecalciferol), and vitamin D4 (22,23-dihydroxyvitamin D2). Chemically, vitamin D is a secosteroid, which is a type of steroid with a broken ring backbone. Vitamin D2 and D3 are the two main types of vitamin D, both of which can be found in our diet in various foods and plants. Vitamin D3 is produced in the skin of animals/humans when ultraviolet (UV) light transforms the cholesterol precursor, 7-dehydrocholesterol, into cholecalciferol. Vitamin D2 is also produced via this reaction in plants, sunlight transforming ergosterol to ergocalciferol.

Vitamin D is indicated for the treatment of hypoparathyroidism (at a dose of 50 μg/kg/day), and refractory rickets (vitamin D resistant rickets) at doses ranging from 3.75-15 mg (150,000 to 600,000 international units “IU”), given intermittently over several months or as a single oral dose. Vitamin D (as cholecalciferol) is commonly used as a supplement in the management of vitamin D deficiency.

The rationale for Vitamin D's use in SARS-CoV-2 infection as a prophylactic is based primarily on evidence showing that vitamin D may be an effective medication to prevent the occurrence and/or severity of respiratory infections.

Vitamin D receptors (VDRs) are highly expressed on B cells and T cells and mediate innate and adaptive immune responses. Vitamin D receptor signaling has been shown to inhibit B and T cell proliferation and differentiation, and it facilitated a change in T cells away from a T helper type 1 (Th1) to a T helper type 2 (Th2) phenotype. Thus, it lowers humoral (B-cell) cytotoxic responses, promotes cytokine production by Th2 cells, and enhances indirect suppression of Th1 cells, resulting in lower acquired immune response. VDR signaling reduces this immune response by inhibiting the production of cytokines by monocytes and dendritic cells, producing anti-inflammatory effects. For example, vitamin D has been shown to reduce production of various pro-inflammatory cytokines (e.g., IL-1, IL-6, IL-8, IL-12, IL-17, IL-21, tumor necrosis factor-α and interferon-γ) and upregulate anti-inflammatory cytokines (IL-10).

Additionally, vitamin D is known to increase nitric oxide release, lysosomal enzyme activity, and Toll-like receptor expression, which enhances the innate immune response and may produce antiviral effects. Studies have also found that vitamin D induces antimicrobial activity by regulating the expression of genes encoding the antimicrobial peptides, cathelocidin and beta defensin 4. These peptides produce direct antimicrobial effects and indirect immunomodulatory effects, such as shifting the adaptive immune response from a Th1 to Th2 phenotype. Vitamin D is known to target vitamin D3 receptor (0.21 nM) and Glycine receptor (400 nM).

Vitamin D3 is readily absorbed from the small intestine. After absorption via chylomicrons, it binds weakly to α-globulin (vitamin D-Binding Protein [DBP]) with a protein binding of 50-80%. It is distributed throughout most tissues and accumulates in the liver within a few hours. There exists a period of 10 to 24 hours between the administration of cholecalciferol and the initiation of its action in the body due to the necessity of synthesis of the active vitamin D metabolites in the liver and kidneys.

### Vitamin E, Alpha Tocopheryl Acetate, and Alpha Tocopheryl Succinate

The vitamin E family encompasses eight lipid-soluble compounds, four tocopherols (α, J3, γ, δ) and four tocotrienols (α, β, γ, δ). Vegetable oils are the main dietary source of vitamin E with soybean, corn, walnut, cottonseed, palm, and germ oils containing higher amounts than other oils. Though γ-tocopherol is the main form of vitamin E in our diet, α-tocopherol is the major form in circulation and has the highest biological activity. The chemically stable form, and the form typically used in supplementation, α-tocopheryl acetate, is hydrolyzed to α-tocopherol and acetic acid following ingestion.

Vitamin E (α-tocopherol) is most commonly used in the treatment of vitamin E deficiency, which can occur due to a poor diet or from chronic diseases including cystic fibrosis, cholestasis, liver disease, and abetalipoproteinemia. Vitamin E has been used in cardiovascular disease, diabetes, cancer, infections, sickle cell anemia, tardive dyskinesia, Alzheimer's Disease, amyotrophic lateral sclerosis, muscle spasms, and retinopathy of prematurity.

Vitamin E is usually administered orally, but it has also been given via intramuscular (IM) or intravenous (IV) routes. It may be given as d-α-tocopherol, or racemic, dl-α-tocopherol, and as the acetate or succinate salts. A proposed oral dose of 300 IU/day vitamin E was identified regarding treatment of COVID-19. A clinical study evaluating multi-vitamin therapy (vitamins A, B, C, D, and E) proposed a twice-daily oral dose of vitamin E at 300 IU (600 IU/day).

Vitamin E is known to target glutathione S-transferase Pi (500 nM). The vitamin E group of molecules, tocopherols and tocotrienols, are fat-soluble antioxidants, protecting cells from oxidative stress, which may be increased due to viral infections. It is well-established in the literature that α-tocopherol exhibits both antioxidant and immunostimulatory effects. Such a combination of effects may reduce the intensity and severity of inflammation and injury stemming from infection of COVID-19.

### Folic Acid

Folic acid, also known as folate or vitamin B9, is a member of the vitamin B group. Humans are unable to synthesize folic acid endogenously; however, it can be found in the diet (e.g., green vegetables, beans, avocados, some fruits, liver, kidney) or be taken as a supplement. *DFE=dietary folate equivalents where 1 μg DFE=1 μg folate from natural sources.

In a randomized controlled trial with COVID-19 patients, a nutritional support system (with 5 mg of folic acid in combination with other vitamins or treatments) is being investigated for reducing complications and comorbidities associated with SARS-CoV-2 infection. In a separate Phase 2/3 trial with adult COVID-19 outpatients, folic acid is used as the placebo control.

Folic acid deficiency in animals leads to lymphoid tissue (e.g., spleen, thymus) atrophy and decreases in circulating T-cell numbers. With adequate supplementation, folic acid can support natural killer cell and cytotoxic T-cell activity, which are important factors in antiviral defense mechanisms.

The entrance of SARS-CoV-2 into the cells occurs via viral spike proteins, which are cleaved into S1 and S2 domains. Furin is an enzyme located on the cellular membrane that activates many precursor proteins and is also suggested to assist in the cleavage of the viral spike protein. It is associated with enhancing a number of bacterial and viral infections and can be a promising target for the treatment of COVID-19. It is proposed that folic acid can inhibit furin and block the interaction of the SARS-CoV-2 spike protein, which subsequently blocks viral entry. Therefore, it has been suggested that folic acid could be beneficial for the management of COVID-19-associated respiratory disease in the early stages of infection. In a separate study using computational analyses and probabilistic scoring, high values were attributed to the strong intermolecular interactions between folic acid and furin enzymes. Using molecular docking models, folic acid was also observed to break the strong interactions between the angiotensin-converting enzyme 2 (ACE-2) protein with amino acids of the viral spike protein. In summary, folic acid can significantly reduce the major interactions between enzymatic host proteins and the viral spike protein, which can interfere with viral entry.

In addition to its inhibitory effects on furin and ACE-2, folic acid and its derivatives can inactivate viral protease 3CLpro, which is an essential viral protein used in the replication of all coronaviruses. The 3CLpro protein is known for counteracting the host innate immune response and has become an attractive target in the treatment of COVID-19. Based on another set of molecular docking interactions, folic acid binds tightly with 3CLpro enzyme, which can prevent viral spread by halting the life cycle of the virus.

Folic acid is known to target matrix metalloproteinase-7 (MMP7; 15 μM), matrix metalloproteinase-9 (MMP9; 22 μM), 72 kDa type IV collagenase (65 μM), matrix metalloproteinase-14 (MMP14; 80 μM).

### Adenine Derivative Compounds, Such as Adenosine, Nicotinamide Adenine Dinucleotide, [Oxidized](NAD+), and Nicotinamide Adenine Dinucleotide [Reduced](NADH)

Adenine is a purine base; adenosine is a nucleotide made of adenine, ribose or deoxyribose, and phosphate groups. NAD is a dinucleotide consisting of two nucleotides joined through their phosphate groups. One nucleotide contains an adenine nucleobase and the other nicotinamide.

Adenine, also known as vitamin B4, is a purine base (component of DNA and RNA) that is used for treating dietary shortage or imbalance. Adenine is not available in a supplemental form; however, doses of 800-1800 mg/day for up to 16 days have been used for the intravenous (IV) administration of oxidized nicotinamide adenine dinucleotide (NAD+) (FDA, 2017). NAD+ and reduced nicotinamide adenine dinucleotide (NADH) are coenzymes composed of ribosylnicotinamide 5′-diphosphate coupled to adenosine 5′-phosphate by a pyrophosphase linkage that serves as an electron carrier by alternating between oxidized (NAD+) and reduced (NADH).

Two studies (one Phase 2 and one Phase 2/3) have been identified for adenine-based nutraceuticals for the treatment or management of COVID-19 symptoms. Oral supplementation (up to 1 g/day for 2 weeks) with an adenine-based nutraceutical is under investigation for the attenuation of SARS-CoV-2 infections in elderly patients. In the second study, the clinical efficacy of metabolic cofactors (including an adenine-based nutraceutical) and other drugs (e.g., hydroxychloroquine) in combination for the treatment of COVID-19 is under investigation (dosing regimen unspecified). One Phase 2 clinical trial has been identified for NAD+(administered as a patch containing 400 mg NAD+) for the treatment of patients with post COVID-19 symptoms. Adenosine at inhaled nebulized doses of 9 mg twice daily (corresponding to 18 mg/day) for one week is being investigated in a Phase 2 study for the downregulation of severe pro-inflammatory responses in COVID-19 patients.

Adenine is one of four nucleic acids utilized in DNA and RNA synthesis, and also plays an important role in healthy tissue development and immunomodulation. Primary dietary sources for adenine include whole grains, vegetables, herbs, and fruits (e.g., apples, oranges, bananas).

NAD+/NADH and adenosine may have important roles in mediating SARS-CoV-2 infections or COVID-19 symptoms. Adenine derivatives are implicated in the host-mediated defense and/or treatment of COVID-19-related symptoms. Activation of the renin-angiotensin (RAS) signaling pathway following viral entry can lead to the release of reactive oxygen species (ROS), which may overwhelm the body with high levels of oxidative stress that may propagate acute respiratory distress syndrome (ARDS).

Decreases in SARS-CoV-2 RNA load and improved lung function were identified in COVID-19 patients that received inhaled doses of adenosine. A wide range of immune cells (e.g., neutrophils, macrophages, lymphocytes) and endothelial cells express adenosine A2A receptors (A2AR), which exert broad-spectrum anti-inflammatory and anti-thrombotic effects. Activation of the receptor from extracellular adenosine results in the downregulation of NF-κB-mediated inflammatory processes, pro-oxidant production, and expression of adhesion molecules. The migration of neutrophils (early mediators of respiratory distress in COVID-19) into the lung interstitial tissue and alveolar space is also suppressed, as are the endothelial cells whose activation attracts and enables the transendothelial passage of activated neutrophils. These considerations suggest that selective agonists of A2AR may have the potential for blunting the severity of COVID-19-related symptoms.

Increases in cellular ATP can potentially improve the efficiency of the innate and adaptive immune systems as ATP may facilitate the production of anti-viral Type I interferon (IFN) proteins and prime cells towards an antiviral state.

### Beta-Carotene

Beta-carotene, a precursor of vitamin A, is used as a supplement in the treatment of vitamin A deficiency, to reduce the severity of photosensitivity reactions in patients with erythropoietic protoporphyria, and other photosensitivity reactions.

The benefits of a multivitamin micronutrient supplementation containing beta-carotene (at doses of up to 1500 μg/capsule) to support immune health and anti-inflammatory/anti-oxidant conditions in COVID-19 patients is underway in two clinical trials.

Beta-carotene is associated with a reduced risk of some chronic diseases, such as coronary heart disease and cancer, which may be attributed to its anti-inflammatory and antioxidant properties. Beta-carotene acts as a scavenger of lipophilic radicals within cell compartments, is a chelator of oxygen-free radicals, and is an inhibitor of lipid peroxidation pathways. It also plays a major role in enhancing cell-mediated immune responses.

Beta-carotene is well-known for its antioxidant, anti-inflammatory, and immunoregulatory capabilities. Beta-Carotene is known to bind to solute carrier organic anion transporter family member 1B1 (OATP1B1; 617 nM) and OATP1B3 (2950 nM). Potential effects of beta-carotene supplementation in the context of COVID-19 may include enhancing the cellular and humoral immune response, the number of T-cell subsets (e.g., promotion of natural killer cell activity), the lymphocyte/leukocyte response to mitogens, interleukin-2 production, and a decrease in NF-κB-mediated cytokine production.

A deficiency of vitamin A is associated with an increased risk of infectious morbidity and mortality in relation to gastrointestinal and respiratory infections.

Increases in natural killer cells and activated lymphocytes were observed in infected patients that received 60 mg of beta-carotene daily for four months. No clinical toxicity was observed. Overall, beta-carotene can be considered a potential anti-inflammatory and immunomodulatory agent for viral infections.

### Calcium

Elemental calcium is a homogeneous alkaline earth metal, and the calcium ion (Ca2+) is an inorganic compound that plays a vital role in the anatomy, physiology and biochemistry of all eukaryotic organisms. Calcium is the most abundant mineral in the body and is an essential body electrolyte. The skeleton acts as a major mineral storage site for calcium, as bone contains 99% of the total body calcium. Calcium is released from bone as ions into the bloodstream under controlled conditions, and circulating calcium is either in the free, ionized, and metabolically active form, or it is bound to blood proteins such as serum albumin.

Calcium, given as various calcium-containing salts, is primarily used in the management of hypocalcemia and calcium deficiency resulting from low calcium intake in the diet or ageing (i.e., osteoporosis). As calcium is essential for the development and maintenance of normal bone, calcium salts may be indicated in the treatment of some bone disorders associated with calcium deficiency, such as certain types of osteomalacia and rickets.

There is evidence that low serum calcium (i.e., hypocalcemia; defined in one study as a serum calcium concentration <2.2 mmol/L) is associated with increased severity of COVID-19 symptoms and poor clinical outcomes. In a meta-analysis of 5 studies, with a total sample size of 1,415 COVID-19 patients, there was a statistically significant decrease in the concentration of serum calcium in patients with severe COVID-19 compared to patients with mild or moderate forms of COVID-19. In these studies, serum sodium and potassium (but not chloride) levels were also significantly decreased in severe COVID-19 patients, suggestive of major ion imbalances in the body.

Results of one study showed that COVID-19 patients with serum calcium concentrations ≤2.0 mmol/L were associated with a significantly higher mortality rate. The 28-day mortality rate was 4.1% overall (in patients with what the physicians defined as mild, moderate, severe, or critical COVID-19), whereas the 28-day mortality of critically ill patients was 40.0%. Serum calcium levels were significantly lower in critical and severe COVID-19 patients, which also exhibited MODS (multiple organ dysfunction syndrome) and septic shock, compared to patients with mild and moderate COVID-19. In summary, the results from this study suggest that low serum calcium is an indicator of the severity of COVID-19 symptoms and may be utilized as a clinical biomarker and/or prognostic.

The hypocalcemia (low serum calcium levels) observed in severe and critical COVID-19 patients was significantly correlated with decreases in serum albumin and vitamin D (i.e., vitamin D deficiency).

One specific mechanism by which calcium may help the clinical outcomes of COVID-19 patients is by binding circulating unsaturated fatty acids (UFAs), which are elevated in obesity and Type 2 diabetes and have been shown to contribute to multiple system organ failure (MSOF). Specifically, abnormally high UFAs have been shown to cause lipotoxicity, resulting in acute lung injury, vascular leak (resulting in decreased albumin levels), inflammatory injury, and cardiac arrhythmias. Notably, this study found that UFA intake (and high serum levels) was positively associated with COVID-19 mortality.

Therefore, some embodiments of the present invention relate to providing calcium to COVID-19 infected patients as part of an attempt to regain or maintain homeostasis of the infected patient.

### Ginkgo Biloba Extract, Ginkgolide a, Ginkgolide B, Ginkgolide C, and Bilobalide

Ginkgo Biloba leaves contain several active alkaloids including terpene trilactones (TTLs), flavanol glycosides, and proanthocyanidins. A standardized and commercially available Ginkgo Biloba Extract (EGb 761®; “GBE”) contains ˜24% flavanol glycosides (primarily quercetin, kaempferol and isorhamnetin) and 6% TTLs (˜3% bilobalides and ˜3% ginkgolides A, B, and C).

GBE has been investigated in clinical studies for the treatment of cerebrovascular and peripheral vascular disorders and neurodegenerative diseases including dementia and Alzheimer's disease.

The pharmacological mechanism of action of ginkgolides resides in the fact that they are antagonists of the platelet activating factor (PAF) receptor. Ginkgolide B reported to have the highest affinity and inhibitory activity. By antagonizing PAF, ginkgolides can decrease platelet activation and aggregation, preventing clotting and adverse vascular events such as thrombosis. This is the reason as to why PAF receptor antagonists have been investigated as treatments for various inflammatory and cardiovascular diseases.

To assess the efficacy of ginkgolides, vasodilation and improvement in blood flow through arteries, veins and capillaries has been observed with GBE use in patients. In a clinical study, oral treatment of GBE for 6 weeks caused vasodilation in forearm blood vessels and increase regional blood flow without alteration in the blood pressure level. Several other studies have indicated that ginkgolides can protect against ischemia and cerebrovascular and traumatic brain injury, as well as inflammation. They function as scavengers of free oxygen radicals, interfere with postischemic production of free oxygen radicals, and decrease glutamate-induced damage of neuronal and hippocampal cells. Overall, treatment with GB may produce anti-platelet, vasodilation, anti-ischemic, and anti-oxidant effects.

Studies have indicated that two major neurotransmitters in the brain, glutamate and γ-aminobutyric acid (GABA) are modulated by bilobalides, which are known GABAA receptor antagonists. Bilobalides are thought to be responsible for the anti-convulsant effects and potentially the neuroprotective effects of Ginkgo Biloba as well. Bilobalides also inhibit phospholipase A2 (PLA2) activity in the brain, resulting in a decrease in hypoxia-induced choline influx.

Anti-inflammatory Actions of Ginkgo Biloba: In addition to the anti-platelet, hypotensive, anti-ischemic effects, the alkaloids in Ginkgo Biloba also exhibit anti-oxidant and anti-inflammatory effects, hypolipidemic, anti-diabetic, and anti-obesity effects.

In preclinical studies, the protective effects of against Lipopolysaccharide (LPS)-induced acute lung injury were demonstrated in rodents. In rats, once daily dosing of GBE for one week prior to LPS-induced acute lung injury was able to decrease the number of inflammatory cells and activities of TNF-α, lactate dehydrogenase, and myeloperoxidase in lung tissue. In the LPS-induced acute lung injury study in mice, histopathological damage, arterial blood gas exchange, overactive inflammatory response, pulmonary edema, and hyaline membrane formation were all improved with GBE treatment. The authors suggested that GBE produced such protective effects by reducing the production of proinflammatory cytokines and chemokines (e.g., TNF-α, IL-6, IL-1, and MIP-2), reducing the expression of COX-2 and nitric oxide (NO), and down-regulating the NF-κB, P38 MAPK, and AMPK signaling pathways.

In human coronary artery endothelial cells, it was shown that ginkgolide A antagonized the release of various inflammatory mediators. Notably, in patients with metabolic syndrome who received GBE treatment, decreased levels of IL-6 were observed, providing further support for the anti-inflammatory properties of ginkgolides.

There is evidence that suggests ginkgolides may have antiviral properties. Ginkgolic acids (i.e. a mixture of several 2-hydroxy-6-alkylbenzoic acids in which the most common alkyl chains contain 13, 15, or 17 carbons) inhibited Herpes simplex virus type 1 by inhibition of both fusion and viral protein synthesis. This study also reported that ginkgolic acids inhibited human cytomegalovirus replication and Zika virus infection. There was broad spectrum inhibition of all three classes of viral fusion proteins in viruses such as HIV, Ebola, influenza A, and Epstein Barr. In addition, ginkgolic acids inhibited a non-enveloped adenovirus, collectively showing that they may inhibit viral replication, fusion, and/or entry during the process of viral infection.

GBE is known to target the platelet activating factor receptor (643 nM). Moreover, a recent paper used a crystal structure of the COVID-19 protease and molecular docking to model and predict inhibition of the protease by various compounds. Ginkgolide A was predicted to have a high binding affinity and therefore demonstrates a potential inhibitory activity against the novel SARS-CoV-2 virus.

In addition to its putative antiviral effects, ginkgolides may help treat the symptoms associated with COVID-19. In patients who died from COVID-19, post-mortem analysis of the lungs showed distinct vascular features consisting of severe endothelial injury, widespread pulmonary thrombosis with microangiopathy, and angiogenesis (i.e., new blood vessel growth).

Given the key role of platelets in the pathogenesis of severe COVID-19, a potent PAF receptor antagonist, such as ginkgolide B, could effectively decrease the pathological effects of platelets. Platelets release PAF and trigger perivascular mast cell activation, leading to significant inflammation; mast cells are one of the main sources of proinflammatory cytokines, especially IL-6. Mast cell degranulation with interstitial edema and thrombosis was observed in the alveolar septa of deceased patients with COVID-19. By decrease PAF-mediated platelet activation and aggregation ginkgolide treatment could decrease the inflammatory response (e.g., decreased mast cell activation, proinflammatory cytokine release) and “cytokine storm” seen in COVID-19.

In a clinical study, GBE at 240 mg/day for 60 days reduced the total mortality risk of cardiovascular disease (CVD) in patients with metabolic syndrome (i.e., type 2 diabetes mellitus). Though GBE did not significantly decrease blood pressure in a clinical study with 3069 elderly subjects, suggesting a lack of efficacy in this specific population, GBE treatment may be beneficial to COVID-19 patients with CVD and/or type 2 diabetes co-morbidities, based on its hypolipidemic effects. Additionally, ginkgolides produce anti-oxidant and anti-ischemic effects that may help in decreasing the extent of lung injury and damage associated with SARS-CoV-2 infection.

### Assessment of Nutraceutical Components

The nutraceutical components (vitamins, minerals, and other pharmacologically active alkaloids) described above were evaluated for their potential in preventing infection with SARS-CoV-2 and treating one or more symptoms of SARS-CoV-2 infection.

The evaluation took into consideration artificial intelligence scores, mechanism of action and pharmacological effects, and clinical and physiological effects including anti-viral, anti-inflammatory, anti-oxidant, anti-platelet, and other immune system modulatory effects.

Nutraceuticals were then ranked based on these factors to identify (1) a potential treatment to prevent or protect against SARS-CoV-2 infection and (2) a potential “post-infection” treatment of COVID-19 symptoms for patients who have tested positive for SARS-CoV-2 infection.

### Protein-Protein Binding

An assessment of various nutraceutical compounds to bind with COVID-19 virus particles was carried out. Using three different mechanism potential binding sites for small molecules, the likelihood of protein-protein binding was determined. Using a template of the crystal structure of an essential SARS-CoV-2 protease, the functional centers of the protease inhibitor-binding pocket were identified.

The DeepDrug™ computational Artificial Intelligence (AI) system was used to assess various of the nutraceutical compounds as likelihood to be effective against SARS-CoV-2 based on the similarity of the drugs to antiviral peptides (AVPs) known to target SARS-CoV-1 and other viruses. AVPs known to inhibit the SARS virus were used as targets. By creating a fingerprint (embedding) of the AVPSs, the AVPs were then compared to similarly generated fingerprints of other known compounds to identify the ones most closely related.

The AVPs used targeted three specific mechanisms: Entry, Fusion, and Replication. The most effective peptides were specifically filtered out and those were used to create three separate networks based on each peptide's known mechanism of action. This allowed the identification of compounds with certain specificities based on mechanism.

The three mechanisms are relevant for the following reasons. Entry is extremely important because inhibiting viral entry into the cell would reduce the amount of virus that acts on the cell. Likewise, inhibition of replication is important for reducing the amount of viral load generated and spread to other cells after a cell has been infected. Finally, fusion though technically least relevant is worth noting because not all viral entry happens through the standard mechanism. The virus is capable of fusing directly with the membrane of the cell for infection. Though this happens at about 1/10th the rate of the standard entry mechanism, it is still a mechanism which was desirable to use as a focus to attempt to inhibit.

The fingerprints of these specific peptides were created by using the human proteome and a large graph of the proteins involved in all the processes therein. By then comparing these fingerprints to the drug fingerprints, the identification of drugs with a similar (antiviral) effect on the human proteome as the AVPs was carried out.

### First Binding Mechanism

A number of nutraceutical compounds where studied to determine their propensity to bind to COVID-19 particles according to a first binding mechanism. The interactions where further evaluated by assessing the likelihood the therapeutic compounds would impact the entry of SARS-CoV-2 virus into mammalian cells; the fusion of SARS-CoV-2 virus particles with mammalian cells; and ultimately the replication of the SARS-CoV-2 virus infected cells. Table 1 summarizes the data obtained in this first round of modeling data analysis.

TABLE 1
Results of Protein-Protein modeling data which mimics
a first mechanism of interaction between SARS-CoV-2
virus and the identified nutraceutical compounds.

| | AI Network MLP (>0.25 is a favorable score) | | | | |
|---|---|---|---|---|---|
| | | | | | Entry, Fusion |
| | Corona | Entry | Fusion | Replication | and/or replication |
| Vitamin D | 0.8389 | 0.9127 | 0.1825 | 0.5028 | Entry and |
| | | | | | replication |
| Vitamin E | 0.0063 | 0.0034 | 0.0038 | 0.0016 | |

According to the data collected in the study of the first binding mechanism, Vitamin D demonstrated a propensity to bind to SARS-CoV-2 virus particles.

### Second Binding Mechanism

With respect to a second binding mechanism, the same two nutraceutical compounds were subsequently studied to determine their propensity to bind to SARS-CoV-2 virus particles. The interactions where also further evaluated by assessing the likelihood the therapeutic compounds would impact the entry of SARS-CoV-2 virus into mammalian cells; the fusion of SARS-CoV-2 virus particles with mammalian cells; and ultimately the replication of the SARS-CoV-2 virus infected cells. Table 2 summarizes the data obtained in this second round of modeling data analysis.

TABLE 2
Results of Protein-Protein modeling data which mimics
a second mechanism of interaction between SARS-CoV-2
virus and the identified nutraceutical compounds.

| | AI Network Snet (<0.5 is an unfavorable score) | | |
|---|---|---|---|
| Corona | Entry | Fusion | Replication |

| | | | |
|---|---|---|---|
| Vitamin D | 0.2733 | 0.2342 | 0.3154 |
| Vitamin E | 0.4701 | 0.466 | 0.4416 |

### Third Binding Mechanism

With respect to a third binding mechanism, the same therapeutic compounds were again subsequently studied to determine their propensity to bind to COVID-19 particles. The interactions where also further evaluated by assessing the likelihood the therapeutic compounds would impact the entry of COVID-19 into mammalian cells; the fusion of COVID-19 particles with mammalian cells; and ultimately the replication of the COVID-19 infected cells. Table 3 summarizes the data obtained in this third round of modeling data analysis.

TABLE 3
Results of Protein-Protein modeling data which mimics
a third mechanism of interaction between SARS-CoV-2
virus and the identified nutraceutical compounds.

| | AI Network Cos Sim (higher = better) | | |
|---|---|---|---|
| | Entry | Fusion | Replication |
| Vitamin D | 0.68794946 | 0.49424823 | 0.68203843 |
| Vitamin E | 0.38643851 | 0.32396994 | 0.36782532 |

### Summary of the Findings

Table 4 provides a summary of the findings with respect to the nutraceutical compounds that were studied and categorizes them into inhibitors of entry, replication or ambiguous or neither.

TABLE 4
Summary of the identified compounds results from AI assessment

| Inhibitors | Inhibitors | Inhibitors of | Ambiguous or |
|---|---|---|---|
| of Entry | of Fusion | Replication | Neither |
| Vitamin D | Levomenol | Vitamin D | Zinc (T = 0.13) |
| (E = 0.82; | (F = 0.78; | (R = 0.82; T = 0.89) | |
| T = 0.89) | T = 0.79) | | |
| | | Ginsenoside Rb1 | Vitamin A |
| | | (R = 0.57; T = 0.57) | (T = 0.08) |
| | | Ginsenoside C | Beta Carotene |
| | | (R = 0.4; T = 0.4) | (T = 0.01) |
| | | Adenine | Folic Acid |
| | | (R = 0.29; T = 0.32) | (T = 0.01) |
| | | Adenosine Phosphate | Ginkgolides |
| | | (R = 0.12; T = 0.16) | (T = 0.01) |
| | | | Glycine Betaine |
| | | | (T = 0.01) |
| | | | Vitamin C |
| | | | (T = 0.01) |
| | | | Calcium (T = 0) |

R indicates replication and T indicates Total score.

### In Vitro Cell Testing

### Experimental Design:

For the in vitro studies, all infectious materials were handled using Biosafety Level 3 protocols. Unless otherwise indicated, the SARS-CoV-2 virus used in these experiments was the 2019 Novel Coronavirus, Isolate USA-WA1/2020 (SARS-CoV-2), which was stored at approximately <−65° C. prior to use. The multiplicity of infection (MOI) will be 0.001 TCID₅₀/cell.

Efficacy of the various nutraceutical compounds was tested in African green monkey kidney (Vero E6) cells and/or human lung cells (Calu-3 cells). Each nutraceutical compound was tested individually. Technicians were blinded to the identification of the drug being tested. Testing of levomenol in Calu-3 cells was performed using SARS-CoV-2 (Isolate SARS-CoV-2/Canada/ON).

Various nutraceutical compound concentrations were evaluated in triplicate for efficacy.

Pre-treatment for 24±4 hours prior to virus inoculation followed by treatment immediately after removal of virus inoculum or 2) treatment only with the nutraceutical compound being tested, which was added immediately following removal of virus inoculum. Remdesivir (as a positive control) was added immediately following removal of virus inoculum. For pre-treatment and treatment, wells were overlaid with 0.2 mL DMEM2 (Dulbecco's Modified Eagle Media (DMEM) with 2% Fetal Bovine Serum (FBS) with test articles at various concentrations). Following the 24±4 hour pre-treatment, cells were inoculated at an MOI of 0.001 TCID50/cell with SARS-CoV-2 and incubated for 60-90 minutes. Immediately following the 60-90 minute incubation, virus inoculum were removed, the cells were washed and appropriate wells were overlaid with 0.2 mL DMEM2 (DMEM with 2% FBS with test or control articles) and incubated in a humidified chamber at 37° C.±2° C. in 5±2% CO₂. At 48±6 hours post inoculation, cells were fixed and evaluated for the presence of virus by immunostaining assay.

For immunostaining, after about 48±6 hours, cells were fixed with paraformaldehyde and stained by anti-SARS-2 nucleoprotein monoclonal antibody (available from Sino Biological) followed by peroxidase-conjugated goat anti-mouse IgG (available from SeraCare). Wells were developed using TMB Substrate Solution and the reaction was stopped by adding acid. The ELISA plate was read at 450 nm on a spectrophotometer by an ELISA plate reader. For each well in the ELISA plate, viral inhibition was calculated as the percentage of reduction of the absorbance value in respect of the virus control by the following formula:

$$\% inhibition =$$

$$100 - [ ( A 450 of test article dilution - A 450 of cell control ) /$$

$$( A 450 of virus control - A 450 of cell control ) ] \times 100.$$

The EC₅₀ was defined as the reciprocal dilution that caused 50% reduction of the absorbance value of the virus control (50% A450 reduction).

TABLE 5
Efficacy of Identified Compounds in Vero E 6 cells

| Compound | EC50 | EC100 | comment |
|---|---|---|---|
| Vitamin D3 | 292.4 | Achieved | |
| Ginseng extract | 2.16 uM | Achieved | |
| Remdesivir | 1.15 uM | Achieved | Positive |

Based on published data from three studies (available online: Jeon et al. Identification of antiviral drug candidates against SARS-CoV-2 from FDA-approved drugs, 2020<https://doi.org/10.1101/2020.03.20.999730>, Li et al. Remdesivir Metabolite GS-441524 Effectively Inhibits SARS-CoV-2 Infection in Mice Models, 2020 <https://doi.org/10.1101/2020.10.26.353300> and Wang et al. Remdesivir and chloroquine effectively inhibit the recently emerged novel coronavirus (2019-nCoV) in vitro <https://doi.org/10.1038/s41422-020-0282-0>) remdesivir was chosen as a positive control compound. From these publications, the average effective concentration (EC₅₀) was calculated to be about 3.45 μM with a cytotoxicity (CC₅₀) of greater than 100 μM.

The antiviral activity of remdesivir and the below identified nutraceutical compounds of the present disclosure were evaluated in vitro, using Vero E6 cells and Calu-3 cells that were infected with a SARS-CoV-2 virus, as follows. A pretreatment and treatment regimen was utilized where the cells were incubated with remdesivir or an identified nutraceutical compound for 24 hours, after which cells were inoculated with SARS-CoV-2 at a multiplicity of infection of 0.005 TCID₅₀ (median tissue culture infectious dose) and incubated at about 37° C. in 5% CO₂ for about 60 to 90 minutes. The viral inoculum was then removed and remdesivir or the identified nutraceutical compound was added and incubated for a further 48±6 hours. Antiviral activity was evaluated using n immunostaining assay, which quantifies the virus' cytopathic effect. Inhibition of any cytopathic effect was calculated as percent reduction relative to positive control (remdesivir treated) and negative control (no treatment). The effective concentration (EC₅₀) was defined as the concentration of drug/nutraceutical that caused 50% reduction relative to the remdesivir control (100% inhibition).

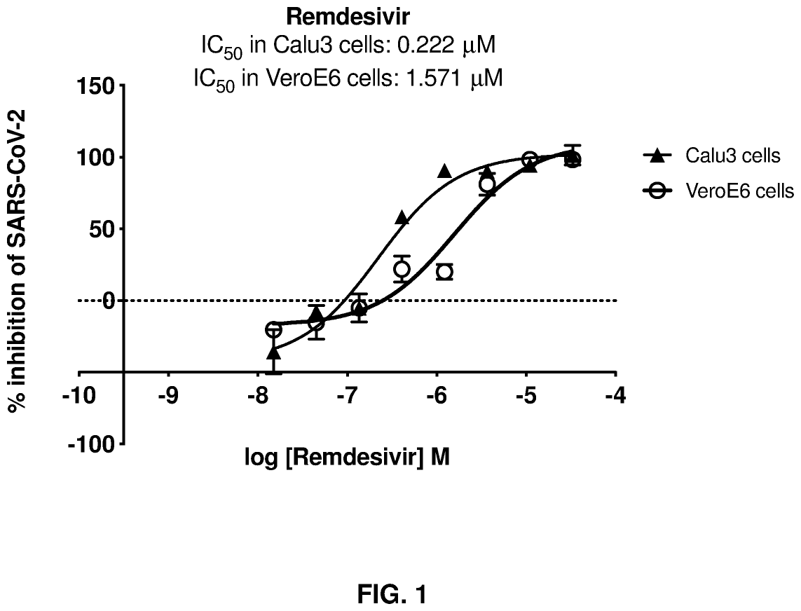

FIG. 1 shows the IC₅₀ results for remdesivir in VeroE6 cells and Calu3 cells.

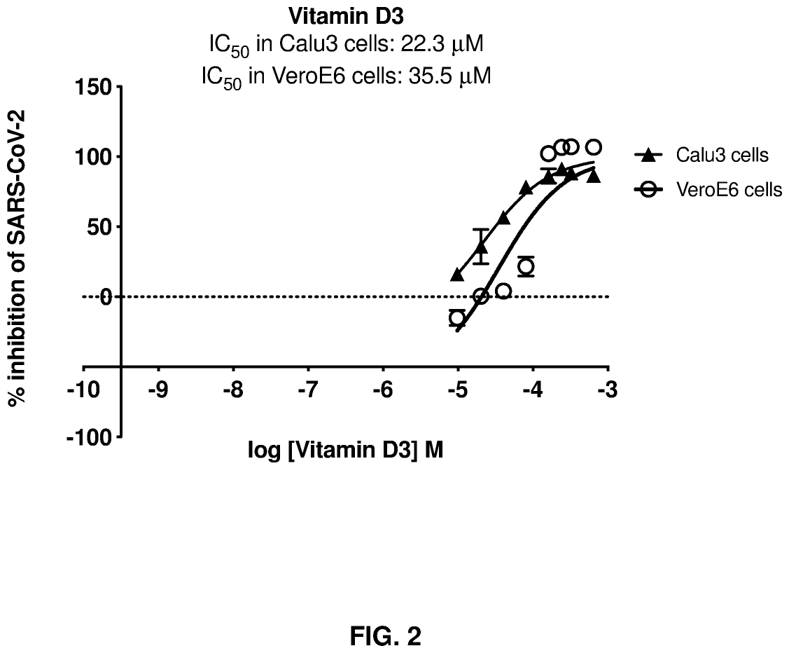

FIG. 2 shows the IC₅₀ results for vitamin D3 in VeroE6 cells and Calu3 cells with about 100% inhibition of the SARS-CoV-2 virus demonstrated and an IC50 of about 33.5 μM in VeroE6 cells and about 22.3 μM in Calu-3 cells. Without being bound by any particular theory, vitamin D is thought to play a role in protecting against pathogens (including viruses), and vitamin D3 deficiency may be a significant risk factor that contributes to SARS-CoV-2 infection and which may be associated with more severe COVID-19 clinical outcomes.

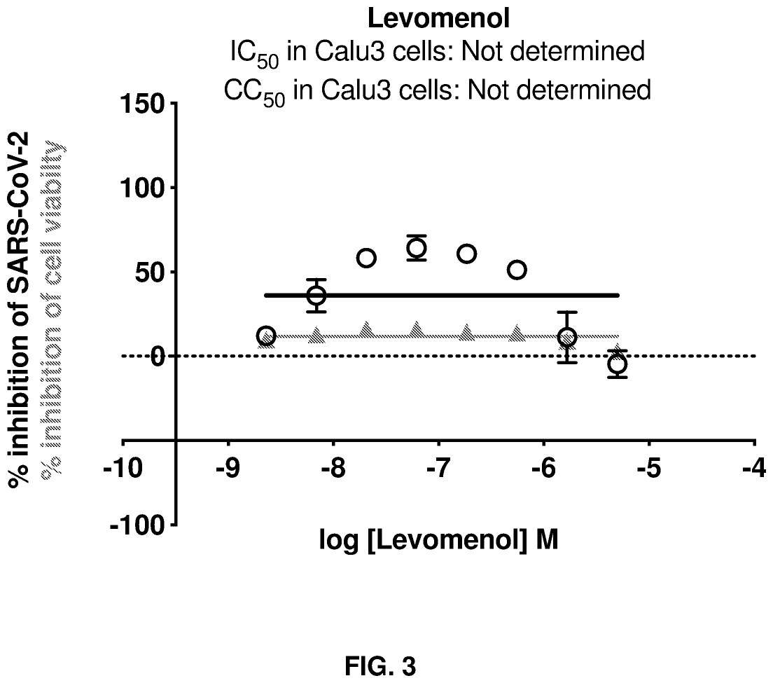

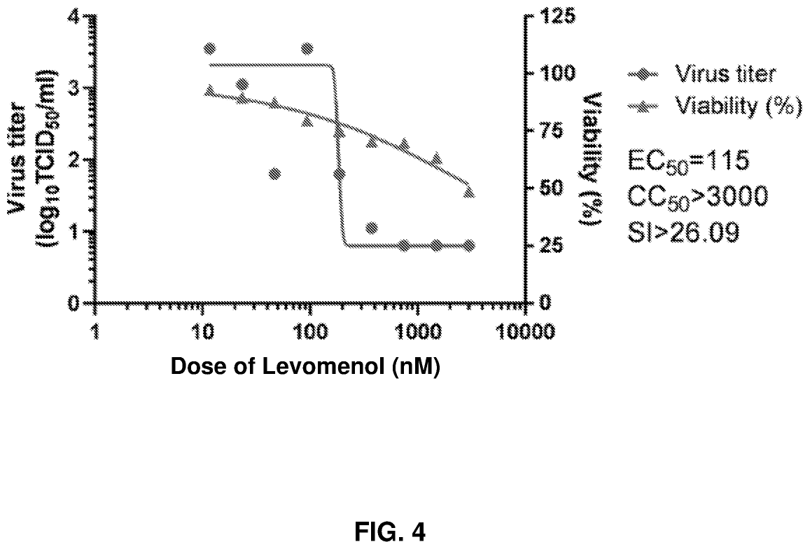

FIG. 3 shows the results for levomenol in VeroE6 cells and Calu3 cells. In Calu-3 cells infected with SARS-CoV-2, about 60% inhibition of the virus was observed following treatment with levomenol. However, an EC50 could not be determined—without being bound by any particular theory, it was postulated that the higher concentrations of levomenol aggregated (crashed out of solution), which interfered with determining the EC50. FIG. 4 shows the virus titer (log 10 TCID50/ml) and cell viability (%) for levomenol treated SARS-CoV-2 infected cells. In Calu-3 cells infected with SARS-CoV-2, levomenol demonstrated antiviral activity against SARS-CoV-2 with an EC50 of 115 nM. Cell viability, a measure of cytotoxicity, decreased by 50% at a cytotoxic concentration (CC50) of 3000 nM. The resulting selectivity index (SI) was calculated to be 26.1 and cytotoxicity, as measured by inhibition of cell viability, was not observed.

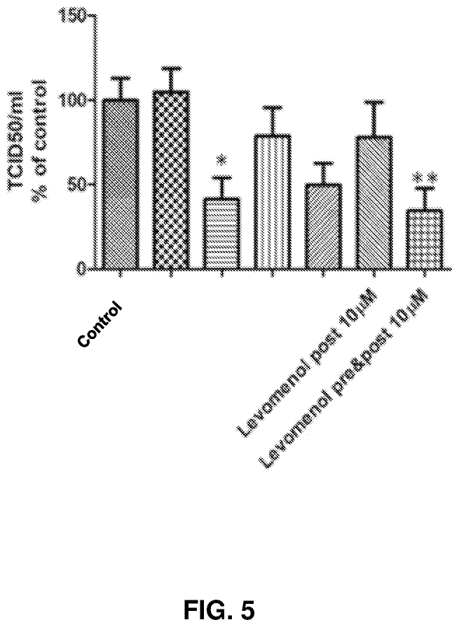

FIG. 5 shows in vitro data of TCID50/ml observed in SARS-CoV-2 infected Vero6 cells treated with levomenol, both after and before and after infection. Without being bound by any particular theory, the before and after treatment with levomenol in this treatment group of infected Vero6 cells further supports the use of levomenol as contemplated herein, when compared to control. Note, the unlabeled data bars are purposefully not identified and do not form part of the present disclosure.

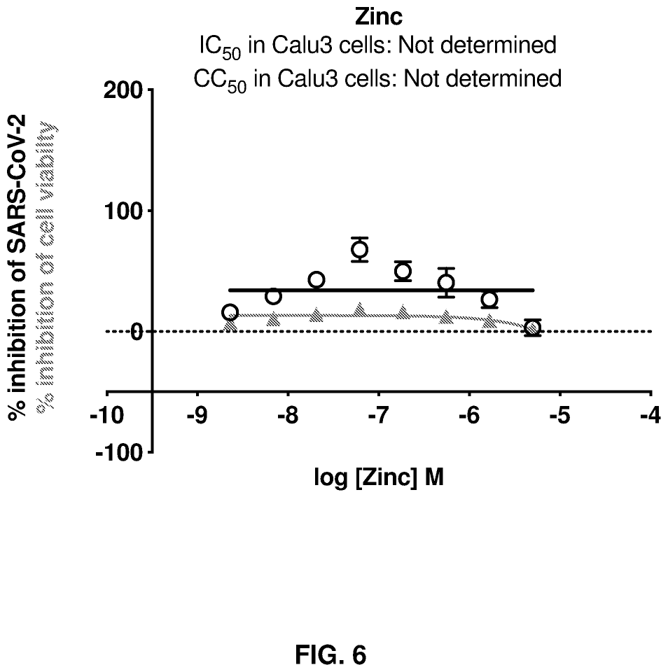

FIG. 6 shows the IC₅₀ results for zinc in VeroE6 cells and Calu3 cells. While the zinc data demonstrated about 75% inhibition of the virus, EC₅₀ was not established and cytotoxicity, as measured by cell viability, was not observed. Without being bound by any particular theory, lower inhibition of SARS-CoV-2 at the higher concentrations may be a result of zinc aggregation (i.e. zinc crashing out of solution).

### Post-Covid-19 Syndrome

One area that is becoming even more concerning to health care workers is the toll Covid-19 and SARS-CoV-2 infections inflict on patients. Some of the symptoms of post-COVID syndrome include: fatigue, difficulty breathing; joint pain; chest pain; brain fog, including an inability to concentrate and impaired memory; loss of taste and/or smell; and sleep issues, to name a few.

According to a preferred embodiment of the present invention, using one or a combination of the nutraceutical compounds discussed above may ameliorate one or more of the symptoms of post-SARS-CoV-2 infection. Ameliorating the symptoms may also result in an accelerated recovery as the nutraceutical compounds may help the recovering subject regain a physiologic balance of vitamins and minerals where such balance of vitamins and minerals was disrupted by the SARS-CoV-2 infection. What is understood by recovery in this context is a substantial improvement over the physical state of an individual when compared to said individual's physical state during the post-Covid-19 infection period and not to said individual's physical state his/her pre-Covid-19 infection. The nutraceuticals according to a preferred embodiment of the present invention not only target COVID-19 but also, in some instances, provide the necessary nutrients to re-balance the body's nutrient imbalances which can last well after some of the more apparent symptoms of COVID-19 have resolved.

Without being bound by any particular theory, use of and methods of administering the formulations of the present disclosure may: reduce or prevent infection with SARS-CoV-2 virus, ameliorate symptoms of a SARS-CoV-2 viral infection, ameliorate one or more of a post-COVID-19 subject's cardiovascular health, antioxidant levels and reduce overall inflammation.

### Formulations of Nutraceutical Compounds

Some embodiments of the present disclosure relate to one or more formulations of the nutraceutical compounds described herein.

Some embodiments of the present disclosure relate to one or more formulations that comprise at least two nutraceutical compounds, as described herein above, that are suitable for providing said compounds to subjects for preventing and/or treating a SARS-CoV-2 infection or the after effects thereof.

In some embodiments of the present disclosure, the formulation may be contained within a capsule delivery form or it may be in a pill or hard tablet delivery form. Depending on the delivery form, the formulation may further include known excipients that are suitable for the selected delivery form.

In some embodiments of the present disclosure, one formulation comprises a ginseng extract from Panax ginseng and levomenol. In some embodiments of the present disclosure one formulation comprises a ginseng extract (Panax ginseng), levomenol and zinc. In some embodiments of the present disclosure one formulation comprises a ginseng extract (Panax ginseng), levomenol, zinc and vitamin D3.

Table 6 below summarizes the constituent components of one formulation that relates to the embodiments of the present disclosure.

TABLE 6
Constituent components of one formulation.

| Ingredients | Amount (mg) | Percentage (%) |
|---|---|---|
| Ginseng Extract (Panax ginseng) | 400.00 | 44.94% |
| Levomenol | 400.00 | 44.94% |
| Zinc Stearate | 50.00 | 5.62% |
| Vitamin D3 1,000 IU | 40.00 | 4.49% |
| TOTAL (g/serving) | 890.00 | 100.00% |

Table 7 below summarizes the constituent components of another formulation that relates to the embodiments of the present disclosure.

TABLE 7
Constituent components of another formulation.

| | | |
|---|---|---|
| Asian Ginseng Extract | ≥400 | mg per serving |
| (Panax ginseng) 100% | | |
| German Chamomile Extract | ≥400 | mg per serving |
| (Levomenol) 100% | | |
| Calcium Carbonate (30%) | ≥400 | mg per serving |
| Vitamin C (Ascorbic Acid) | ≥150 | mg per serving |
| Zinc Oxide | ≥50 | mg per serving |
| Biotin | ≥45 | mg per serving |
| Vitamin E 28 IU | ≥12.6 | mg per serving |
| Beta Carotene 1000 IU | ≥600 | μg per serving |
| Folic Acid | ≥400 | μg per serving |
| Vitamin A Acetate 1000 IU | ≥300 | μg per serving |
| Vitamin D3 1,000 IU | ≥200 | μg per serving |

Optionally, either of the two formulations above may also include between about 1 to 5 μg of vitamin B12.

The person skilled in the art will recognize that the precise amounts of each component in the two formulations above may be varied while still contemplated by this disclosure. Either of the two formulations above may provide a daily therapeutically effective amount of the nutraceutical compounds therein when taken as a single delivery unit of the formulation, once, twice or more times per day.

For example, the total amount of levomenol (% weight of the total weight of the formulation) may be between about 15% and about 85%, or between about 20% and about 80%, or between about 30% and about 70% or between about 40% and about 60%. In some embodiments of the present disclosure the total amount of levomenol in the formulation is between about 40% and 50%.

The total amount of a ginseng extract (for example, Panax ginseng) may be between about 15% and about 85%, or between about 20% and about 80%, or between about 30% and about 70% or between about 40% and about 60%. In some embodiments of the present disclosure the total amount of the ginseng extract in the formulation is between about 40% and 50%. The ginseng extract may comprise one or more gingenoside compounds.

The total amount of zinc (for example in the form of a zinc salt) may be between about 1% and about 10%, or between about 2% and about 9%, or between about 3% and about 8%, or between about 4% and about 7%, or between about 5% and about 6%. The total amount of vitamin D (for example vitamin D3) may be between 1% and about 10%, or between about 2% and about 9%, or between about 3% and about 8%, or between about 4% and about 7%, or between about 4.5% and about 6.5%.

In some embodiments of the present disclosure, the total amount (by weight) of levomenol per delivery unit of the formulation may be between about 200 mg and about 600 mg, or between about 250 mg and about 550 mg, or between about 300 mg and about 500 mg, or between about 350 mg and about 450 mg. In some embodiments of the present disclosure, the total amount (by weight) of levomenol is greater than about 400 mg.

In some embodiments of the present disclosure, the total amount (by weight) of a ginseng extract (for example, Panax ginseng) per delivery unit of the formulation may be between about 200 mg and about 600 mg, or between about 250 mg and about 550 mg, or between about 300 mg and about 500 mg, or between about 350 mg and about 450 mg. In some embodiments of the present disclosure, the total amount (by weight) of the ginseng extract is greater than about 400 mg.

In some embodiments of the present disclosure, the total amount (by weight) of zinc (for example in the form of a zinc salt) per delivery unit of the formulation may be between about 30 mg and about 80 mg, or between about 40 mg and about 70 mg, or between about 50 mg and about 60 mg. In some embodiments of the present disclosure, the total amount (by weight) of zinc is greater than about 50 mg.

In some embodiments of the present disclosure, the total amount (by weight) of vitamin D (for example, in the form of vitamin D3 from a 1000 IU source) may be between about 50 μg and about 500 μg, or between about 100 μg and about 450 μg, or between about 150 μg and about 400 μg, or between about 200 μg and about 350 μg. In some embodiments of the present disclosure, the total amount (by weight) of vitamin D is greater than about 200 μg.

In some embodiments of the present disclosure, either of the two formulations may be ingested by a subject in a single delivery unit (capsule, pill or tablet) once, twice or three times a day (or more) in order to provide a daily therapeutic amount of the indicated nutraceutical compounds. The daily therapeutic amount may be continually taken while the subject is at risk of being exposed to a SARS-CoV-2 virus and/or following exposure to a SARS-CoV-2 virus and/or becoming infected by a SARS-CoV-2 virus.

1. A nutraceutical composition comprising: at least 20 weight % of levomenol and at least 20 weight % of a ginseng extract.

2. The nutraceutical composition of claim 1, further comprising at least 1 weight % of a zinc compound.

3. The nutraceutical composition of claim 1, further comprising at least 1 weight % of a vitamin D compound.

4. The nutraceutical composition of claim 1, wherein there is between 40 weight % and 50 weight % of levomenol.

5. The nutraceutical composition of claim 1, wherein there is between 40 weight % and 50 weight % of the ginseng extract.

6. The nutraceutical composition of claim 1, wherein the ginseng extract comprises one or more gingenoside compounds.

7. The nutraceutical composition of claim 1, further comprising one or more of a ginkgolide compound, vitamin A; vitamin E; vitamin D; vitamin C; and one or more adenine derivative compounds.

8. A method for treating one or more post-Covid-19 symptoms in a subject, the method comprising: administering a combination of levomenol and a ginseng extract to the subject.

9. The method of claim 8, wherein the combination further comprises zinc.

10. The method of claim 8, wherein the combination further comprises vitamin D.

11. The method of claim 8, wherein the combination further comprises one or more of a ginkgolide compound, vitamin A; vitamin E; vitamin D; vitamin C; and one or more adenine derivative compounds.

12. The nutraceutical composition of claim 7, wherein the one or more adenine derivative compounds is chosen from Adenosine, NAD+, NADH, and combinations thereof.

13. The method of claim 8, wherein the combination of the levomenol and the ginseng extract is administered as a nutraceutical composition comprising at least 20 weight % of levomenol and at least 20 weight % of a ginseng extract.
